(19) United States
(12) Patent Application Publication
(10) Pub. No.: US 2025/0052840 A1
(43) Pub. Date: Feb. 13, 2025

(54) MAGNETIC RESONANCE APPARATUS WITH SWITCHING UNIT AND METHOD FOR OPERATING SUCH AN APPARATUS

(71) Applicant: Siemens Healthineers AG, Forchheim (DE)

(72) Inventor: Ralph Kimmlingen, Zirndorf (DE)

(21) Appl. No.: 18/800,061

(22) Filed: Aug. 10, 2024

(30) Foreign Application Priority Data
Aug. 10, 2023 (EP) 23190884.9

(51) Int. Cl. G01R 33/36

(52) U.S. Cl. CPC G01R 33/3614; G01R 33/3664

(57) ABSTRACT

A magnetic resonance apparatus with a switching unit and a method for operating such an apparatus are provided. The magnetic resonance apparatus includes a power amplifier unit that is configured to provide transmit signals to be emitted by the magnetic resonance apparatus, and the switching unit. The switching unit includes an actuator unit, a first plug-in connector part that is connected to the power amplifier unit, and a number of second plug-in connector parts. The actuator unit is configured to move the first plug-in connector part relative to the number of second plug-in connector parts so that an electrical connection is established between the first plug-in connector part and a predetermined plug-in connector part of the number of second plug-in connector parts.

This application claims the benefit of European Patent Application No. EP 23190884.9, filed on Aug. 10, 2023, which is hereby incorporated by reference in its entirety.

### BACKGROUND

The present embodiments relate to a magnetic resonance apparatus with a switching unit and to a method for operating such an apparatus.

In medical engineering, imaging by magnetic resonance (MR), also referred to as magnetic resonance imaging (MRI), is characterized by high soft tissue contrast. A patient (e.g., a human or animal patient) is positioned in a main magnetic field of a magnetic resonance apparatus. During a magnetic resonance measurement (e.g., with the aid of a radio-frequency antenna unit of a magnetic resonance apparatus), radiofrequency (RF) pulses are radiated into the patient. Nuclear spins are excited in the patient by the RF pulses created, whereby spatially-encoded magnetic resonance signals are triggered. The magnetic resonance signals are received by the magnetic resonance apparatus and used for reconstruction of magnetic resonance images.

For exciting signals in a magnetic resonance apparatus with an ultra-high main magnetic field (UHF) (e.g., 7 Tesla or more), local coils (e.g., body region-specific transmit-receive coils) may be used. By contrast with the lower field strengths (e.g., 3 Tesla or less), the use of a body coil has not been able to become established (e.g., on account of its typically poor transmit and SAR efficiency). As a consequence, for an MR measurement with a UHF magnetic resonance apparatus, it is necessary to carry out a change of coil for each measuring region.

In order to connect a local coil, the magnetic resonance apparatus often has one or more local coil connection units on the patient table (e.g., in the form of one or more connector sockets). Depending on which region is to be measured, a corresponding local coil is connected to one of the number of local coil connection units. Each of the number of local coil connection units has a number of transmit or receive channels. In order to satisfy the requirements of workflow and safety, the cable length of a plug-in local coil is chosen to be as short as possible. Therefore, the local coil suitable for a particular measurement is connected to an adjacent local coil connection unit. However, the overall number of transmit or receive channels of the magnetic resonance apparatus is limited, so that the number of local coil connection units is also limited and their spatial distribution over the patient table is restricted.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a workflow during a measurement in which local coils are used is improved. As another example, a number of local coil connection units for connecting local coils, such as transmit-receive coils, in a cost-efficient manner are provided.

Independent of the grammatical term usage, individuals with male, female, or other gender identities are included within the term.

A magnetic resonance apparatus that includes a power amplifier unit that is configured to provide transmit signals to be emitted by the magnetic resonance apparatus, and includes a switching unit is provided. The transmit signals to be emitted are, for example, suitable for a parallel transmission (pTx). The switching unit includes an actuator unit, a first plug-in connector part that is connected to the power amplifier unit, and a number of second plug-in connector parts. In this case, the actuator unit is configured to move the first plug-in connector part relative to the number of second plug-in connector parts (e.g., to position the first plug-in connector part so that an electrical connection, such as a plug-in connection, may be established between the first plug-in connector part and a predetermined plug-in connector part of the number of second plug-in connector parts). For example, the actuator unit is configured to switch over between the first plug-in connector part and the number of second plug-in connector parts.

In one embodiment, with the aid of the switching unit, a specific number of transmit channels available may be switched to a local coil connection unit in each case. For example, the magnetic resonance apparatus includes a number of local coil connection units (e.g., plug-in slots) that are each configured to connect a local coil to the magnetic resonance apparatus. Each of the number of second plug-in connector parts is electrically connected in each case to one of the number of local coil connection units. The present embodiments simplify division of RF transmit power between a number of local coil connection units with a low overhead for additional RF lines and/or switchover contacts.

For example, the magnetic resonance apparatus (e.g., the power amplifier unit) has N (N≥1) transmit channels that are connected to the first plug-in connector part; the switching unit, for example, has M (e.g., M>1) second plug-in connector parts as well as M local coil connection units connected thereto. In one embodiment, each of the M local coil connection units may then be connected to the N transmit channels via the switching unit. Without a switching unit, N×M transmit channels may then be required for this instead of just N transmit channels (e.g., the number of the transmit channels of the power amplifier unit (and the hardware connected thereto such as amplifiers, etc.) may be reduced).

Through the use of plug-in connector parts or a plug-in connection, a connection that has a high immunity to interference, a high switching performance (e.g., required performance range of around 8-32 kW peak), and/or only low losses is made possible. Such plug-in connections are far superior to any semiconductor switches. Any reflections at open ends may further be avoided. In one embodiment, such a proposed plug-in connection is suitable for magnetic fields or is MR-compatible. Further, the proposed switching unit may be implemented at comparatively low cost.

The prespecified second plug-in connector part may be prespecified, for example, by a manual selection by the operating personnel of the magnetic resonance apparatus of a local coil connection unit connected to the second plug-in connector part. Such a selection may be automated, depending on a magnetic resonance examination to be carried out. If, for example, a head examination is to be carried out, a connection to the second plug-in connector part that is connected to the local coil connection unit may be established automatically, which is particularly suitable for connection to a head coil or is best suited to this.

In one embodiment, the first plug-in connector part corresponds mechanically and/or electrically to each of the number of second plug-in connector parts. For example, the first plug-in connector part forms a mating element with each of the number of second plug-in connector parts. For example, the first plug-in connector part is a male connector, and each of the number of second plug-in connector parts is a corresponding female connector.

For example, the first plug-in connector part is a male connector, and the number of second plug-in connector parts are female connectors corresponding to the male connector. For example, the first plug-in connector part is a female connector, and the number of second plug-in connector parts are male connectors corresponding to the female connector.

In one embodiment, the first plug-in connector part and the second plug-in connector part connected to the first plug-in connector part are a system for transmission of information and/or electrical energy. In one embodiment, the transmit signals to be emitted may be transmitted via an electrical connection.

In one embodiment, each of the number of second plug-in connector parts is configured to make an electrical connection to the first plug-in connector part, so that the transmit signals may be transmitted via this electrical connection.

For example, the first plug-in connector part has a first shape (e.g., first geometrical shape), and each of the number of second plug-in connector parts has a second shape (e.g., second geometrical shape). The first shape and the second shape are matched to one another so that, when the electrical connection is established, the first shape and the second shape may be mechanically guided and/or engage into one another.

In one embodiment, the first plug-in connector part and/or the number of second plug-in connector parts have a housing. The shape of the plug-in connector part may, for example, be dictated by the shape of the housing of the plug-in connector part.

In one embodiment, the first plug-in connector part and/or the number of second plug-in connector parts have electrical contact elements corresponding to one another (e.g., electrical contact pins and/or contact openings). In one embodiment, the contact elements are configured (e.g., in the connected state) to form an electrically resilient contact. In one embodiment, an individual contact is configured to transmit a peak power of more than 0.5 kW (e.g., more than 1 kW or more than 1.5 kW). In one embodiment, an individual contact is configured to transmit an average power of more than 50 W (e.g., than 100 W or more than 150 W). In one embodiment, the switching unit contact is configured to transmit a total peak power of more than 8 KW (e.g., more than 12 kW or more than 16 kW). In one embodiment, an individual contact is configured to transmit an average power of more than 0.8 kW (e.g., more than 1.2 kW or more than 1.6 kW).

In one embodiment, the first plug-in connector part and/or the number of second plug-in connector parts have at least one electrical contact pin and/or at least one contact opening corresponding thereto. The shape of the plug-in connector parts may, for example, be dictated by electrical contact pins of the plug-in connector part and/or by their receptacles on the corresponding plug-in connector part. The electrical contact pins may be pointing outwards (e.g., male part of a plug-in connection). The receptacles may be contact openings pointing inwards (e.g., female part of a plug-in connection). A plug-in connector part may simultaneously have both contact pins and also contact openings (e.g., connector elements of both genders).

In one embodiment, the first plug-in connector part and/or the number of second plug-in connector parts include at least one coaxial electrical contact element.

For example, the first plug-in connector part and/or the number of second plug-in connector parts include at least one coaxial contact pin. A coaxial connection may make an especially low-noise signal transmission possible.

In one embodiment, the first plug-in connector part and/or the number of second plug-in connector parts include at least one mechanical guide element.

For example, the first plug-in connector part and/or the number of second plug-in connector parts include at least one mechanical guide pin and/or at least one guide opening corresponding thereto. In one embodiment, the at least one mechanical guide pin is a massive design.

For example, the first plug-in connector part includes a mechanical guide pin, and the number of second plug-in connector parts include a guide opening corresponding to the mechanical guide pin in each case.

In one embodiment, the at least one mechanical guide pin, together with the at least one guide opening corresponding thereto, make possible a precise and/or reliable process of plugging the first plug-in connector part into the respective second plug-in connector part.

In one embodiment, the actuator unit includes at least one actuator. The actuator is, for example, a component that converts a, for example, electrical control signal into a mechanical movement. The control signal may, for example, be transferred from a system control unit of the magnetic resonance apparatus to the actuator unit. In one embodiment, the actuator unit includes an actuator control unit that is configured to receive control signals of the system control unit.

In one embodiment, the system control unit is configured to send the control signals to the actuator control unit depending on a magnetic resonance sequence to be carried out (e.g., to one or more local coils selected for this). If a measurement of the head of the patient is carried out in accordance with the magnetic resonance sequence, for example, a connection of the first plug-in connector part to the second plug-in connector part may be established by the actuator unit, which is connected to the local coil connection unit that is suitable for connection of a head coil.

For example, the actuator unit (e.g., the at least one actuator) includes at least one pneumatic drive. In this case, forces for movement of the first plug-in connector part may be transmitted by a gas (e.g., compressed air).

For example, the actuator unit (e.g., the at least one actuator) includes at least one hydraulic drive. In this case, forces for movement of the first plug-in connector part may be transmitted by a liquid (e.g., an oil, compatible with given standards).

The at least one pneumatic drive and/or the at least one hydraulic drive may be configured to convert pressure energy into movement energy in order to move the first plug-in connector part.

For example, the actuator unit (e.g., the at least one actuator) includes at least one electric motor. The at least one electric motor may be an electromechanical converter that converts electrical power into mechanical power in order to move the first plug-in connector part.

In one embodiment, the at least one electric motor is screened. In one embodiment, interference is reduced by the screening.

In one embodiment, the magnetic resonance apparatus includes a magnet for creation of a main magnetic field. The at least one electric motor has a rotor that interacts with the main magnetic field of the magnetic resonance apparatus as a permanent magnetic field of the electric motor. In one embodiment, the main magnetic field present in any event may thus be used for operation of the electric motor.

In one embodiment, the actuators proposed above are suitable for carrying out a switchover process even in a strong magnetic field.

In one embodiment, the actuator unit includes a roller extension. The first plug-in connector part may, for example, be arranged on a running rail. In this, for example, a transmit line that has a diameter of around 5 mm and a bending radius diameter of around 8 cm is employed. A winding spindle with approximately 10 cm diameter and spring reset force may be provided, for example. Two windings may, for example, suffice for providing around 50 cm of travel for the plug-in connector part.

In one embodiment, the actuator unit is configured to move the first plug-in connector part in at least two spatial directions (e.g., directions of movement). For example, the number of second plug-in connector parts are arranged along a straight line, where this straight line corresponds to a first of the directions of movement of the actuator unit.

In one embodiment, the number of second plug-in connector parts are arranged in one plane, where the actuator unit is configured to move the first plug-in connector part in parallel to the plane in order to bring the first plug-in connector part into the vicinity of one of the number of second plug-in connector parts and/or so as to align the first plug-in connector part with one of the number of second plug-in connector parts.

A second direction of movement of the actuator unit is, for example, a direction at right angles to the first direction of movement, in order, by movement in this direction, to make the connection between the first plug-in connector part and one of the number of second plug-in connector parts and to release the connection again by movement in the opposite direction.

In one embodiment, the number of second plug-in connector parts are arranged in one plane, where the actuator unit is configured to move the first plug-in connector part at right angles to the plane in order to make the connection between the first plug-in connector part and one of the number of second plug-in connector parts and to release the connection again by movement in the opposite direction.

In one embodiment, the switching unit is arranged in the same room in which the magnetic resonance apparatus (e.g., rest of the magnetic resonance apparatus; its magnet unit) is located. In one embodiment, the magnetic resonance apparatus incudes a patient table and a magnet unit for creation of a main magnetic field of the magnetic resonance apparatus. The switching unit is arranged in the area of the patient table and/or in the vicinity of the magnet unit.

The arrangement of the switching unit on the magnetic resonance apparatus may enable short signal lines to be made possible. A possible arrangement of the switching unit in a room adjacent to the room in which the magnetic resonance apparatus is located may by contrast require relatively long signal lines. Thus, even line losses otherwise present may be minimized, so that the dielectric strength requirements on the electrical connections may be greatly reduced.

In one embodiment, the magnetic resonance apparatus has a main magnet with a strength of at least 5 Tesla. With such magnetic resonance apparatuses, local coils (e.g., RF transmit coils) may be employed especially effectively by the local coil connection units.

In one embodiment, the magnetic resonance apparatus includes at least one local coil that is configured to emit radio-frequency pulses in accordance with the transmit signals provided into an examination region of the magnetic resonance apparatus.

A method for providing a correct connection is further provided. In this method, at least one radio-frequency pulse is emitted with a magnetic resonance apparatus previously described, and its amplitude and/or phase reflection is evaluated. In one embodiment, with the aid of the information evaluated, it may be established whether the switching unit has established a connection to the correct second plug-in connector part and to the local coil connection unit connected thereto.

In one embodiment, the at least one radio-frequency pulse is emitted at the beginning of carrying out a magnetic resonance sequence. In one embodiment, this enables a possible error to be established at an early stage.

In one embodiment, the at least one radio-frequency pulse has a small amplitude. In one embodiment, this enables the check to be made especially safely.

The features, advantages, or alternate forms of embodiment of the magnetic resonance apparatus, which have been set out above in detail, may be transferred to the method of the present embodiments for establishing a correct connection and vice versa.

Further advantages, features, and details of the invention emerge from the example embodiments described below, as well as with the aid of the drawings. Parts that correspond to one another are provided with the same reference characters in all figures.

### BRIEF DESCRIPTION OF THE DRAWINGS

FIG. 1 shows one embodiment of a magnetic resonance apparatus;

FIG. 2 shows one embodiment of a switching unit with a row of second plug-in connector parts;

FIG. 3 shows one embodiment of a switching unit with two rows of second plug-in connector parts;

FIG. 4 shows possible arrangements of the switching unit on the magnetic resonance apparatus; and

FIG. 5 shows one embodiment of a method for providing a correct connection.

### DETAILED DESCRIPTION

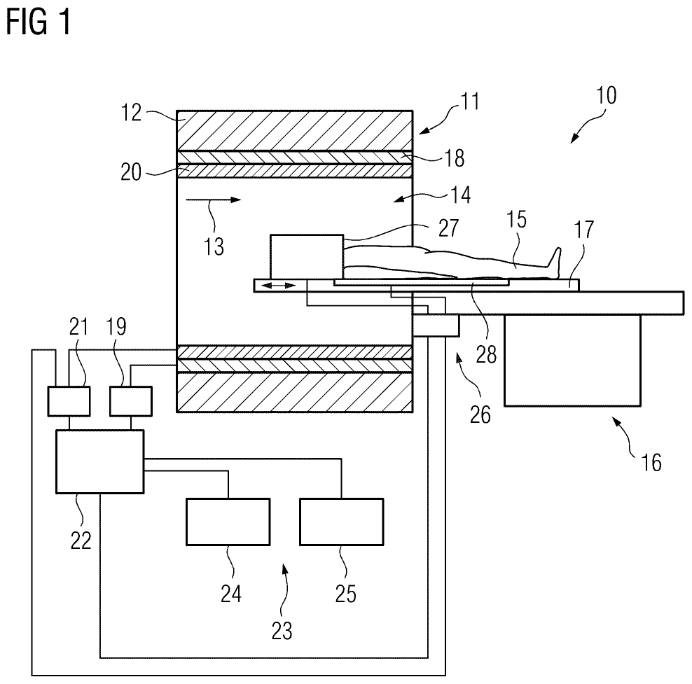

Shown schematically in FIG. 1 is a magnetic resonance apparatus 10. The magnetic resonance apparatus 10 includes a magnet unit 11 that has a main magnet 12 for the creation of a strong and, for example, temporally constant main magnetic field 13. The main magnetic field 13 is, for example, an ultra-high magnetic field. Further, the magnetic resonance apparatus 10 includes a patient receiving area 14 for receiving a patient 15. In the present example embodiment, the patient receiving area 14 is configured cylindrical in shape and is surrounded cylindrically in a circumferential direction by the magnet unit 11. The patient 15 may be pushed by a patient support apparatus 16 of the magnetic resonance apparatus 10 into the patient receiving area 14. For this purpose, the patient support apparatus 16 has a patient table 17 configured movably within the patient support area 14.

The magnet unit 11 also has a gradient coil unit 18 for creation of magnetic field gradients that are used for spatial encoding during an imaging process. The gradient coil unit 18 is controlled by a gradient control unit 19 of the magnetic resonance apparatus 10. The magnet unit 11 also includes a radio-frequency antenna unit 20 that, in the present example embodiment, is configured as a body coil permanently integrated into the magnetic resonance apparatus 10. Unlike those shown here, UHF magnetic resonance apparatuses may not have a body coil permanently integrated into the magnetic resonance apparatus 10; however; instead, local coils specific to regions of the body, such as a head coil 27 or a spinal column coil 28, for example, are used, which are configured to radiate radio-frequency magnetic resonance sequences into an examination space that is essentially formed by a patient receiving area 14 of the magnetic resonance apparatus 10. Through this, the main magnetic field 13 created by the main magnet 12 sets an excitation of atomic nuclei. By relaxation of the excited atomic nuclei, magnetic resonance signals are created. The local coils are, for example, configured both for transmitting RF signals and also for receiving the magnetic resonance signals.

For control of the main magnet 12 and for control of the radio-frequency antenna control unit 21, the gradient control unit 19 and the magnetic resonance apparatus 10 have a system control unit 22. The system control unit 22 centrally controls the magnetic resonance apparatus 10, such as, for example, the carrying out of a predetermined imaging gradient echo sequence. Further, the system control unit 22 has an evaluation unit, not shown in any greater detail, for an evaluation of the magnetic resonance signals that are detected during the magnetic resonance examination. Further, the magnetic resonance apparatus 10 has a user interface 23 that is connected to the system control unit 22. Control information, such as, for example, imaging parameters, as well as reconstructed magnetic resonance images, may be shown on a display unit 24 (e.g., on at least one monitor of the user interface 23 for a medical operator). Further, the user interface 23 has an input unit 25, by which information and/or parameters may be entered by the medical operating personnel during a measurement process.

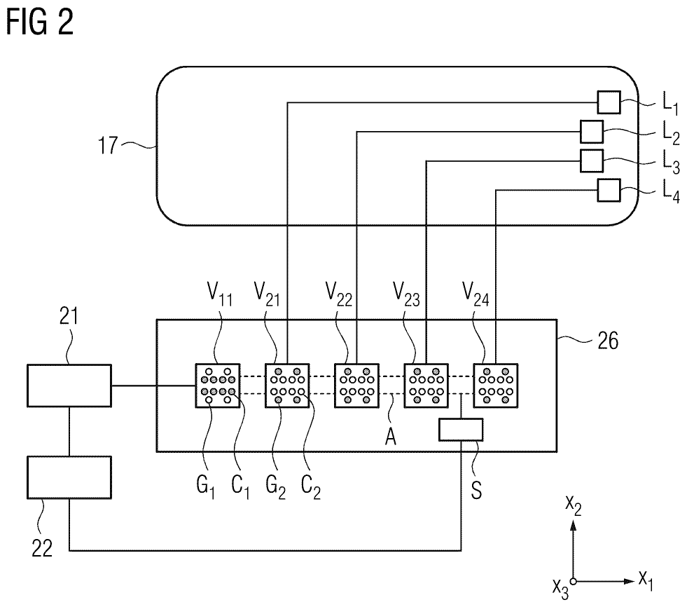

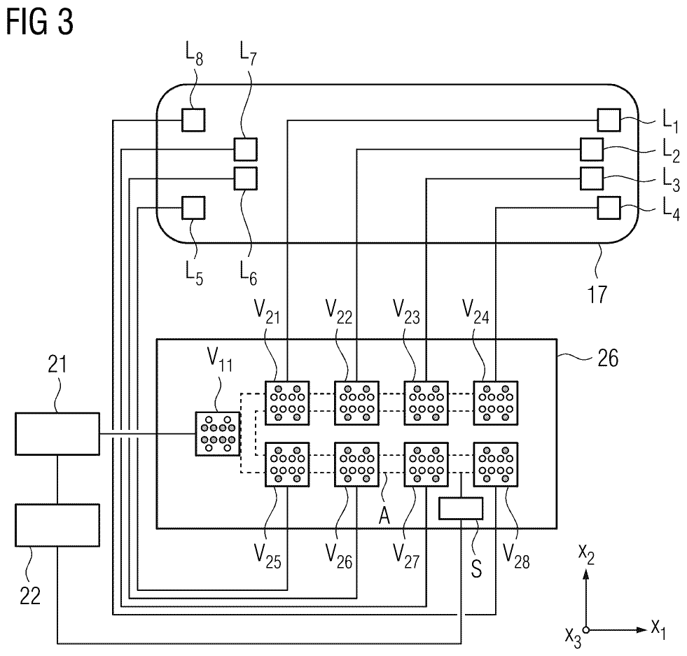

For operation of the local coils, the magnetic resonance apparatus 10 includes a radio-frequency antenna control unit 21 that has a power amplifier unit with a number of transmit channels. The power amplifier unit is configured to provide transmit signals to be emitted in order, for example, to create pTx pulses. In order to switch the available transmit channels, the magnetic resonance apparatus includes a switching unit 26 that is described in more detail in FIGS. 2 and 3. The transmit lines from the power amplifier unit end at a first plug-in connector part of the switching unit (cf. FIGS. 2 and 3).

Shown in FIG. 2 is a switching unit that includes an actuator unit, a first plug-in connector part V₁₁ that is connected to the power amplifier unit of the radio-frequency antenna control unit 21, and four second plug-in connector parts V₂₁, V₂₂, V₂₃, V₂₄. The actuator unit A is configured to position the first plug-in connector part V₁₁ relative to the four second plug-in connector parts so that an electrical connection is established between the first plug-in connector part V₁₁ and one of the number of second plug-in connector parts V₂₁, V₂₂, V₂₃, or V₂₄, that has, for example, been selected beforehand.

This provides that the actuator unit A is capable of moving the first plug-in connector part V₁₁ in the direction ±x₁ along the plug-in connector parts V₂₁, V₂₂, V₂₃, V₂₄ that are arranged along a straight line parallel to direction x₁. The directions x₁, x₂, x₃ in FIGS. 2 and 3 may be orthogonal to one another. Thus, through a movement in this direction, the desired second plug-in connector parts V₂₁, V₂₂, V₂₃ or V₂₄ may be moved too. For example, the first plug-in connector part V₁₁ is over the second plug-in connector part V₂₃ after such a movement.

The actuator unit A is further capable of moving the first plug-in connector part V₁₁ in the direction ±x₃. Through movement in this direction, a connection may be established between the first plug-in connector part V₁₁ and the desired second plug-in connector parts V₂₁, V₂₂, V₂₃ or V₂₄ or also released again. For example, after its arrival above the plug-in connector part V₁₁, the second plug-in connector part V₂₃ is moved downwards into the plug-in connector part V₂₃ in order to establish an electrical connection.

In order to control the movements, the switching unit 26 further includes an actuator control unit S that may receive control signals from the system control unit 22 and convert these into corresponding movements of the first plug-in connector part V₁₁ by the actuator unit A. The system control unit 22 also controls the radio frequency antenna control unit 21 with its power amplifier unit, which in the present case includes eight power amplifiers, one for each transmit channel. Accordingly, the first plug-in connector part V₁₁ also has eight electrical contact elements C₁. Such contact elements may, for example, be electrical contact pins, and corresponding thereto contact elements C₂, which, for example, make an electrical connection in contact openings when the first plug-in connector part V₁₁ is plugged into the desired second plug-in connector parts V₂₁, V₂₂, V₂₃ or V₂₄.

In order to carry out a reliable connection process (e.g., plug-in process), the plug-in connector parts V₁₁ and also V₂₁, V₂₂, V₂₃ or V₂₄ may have guide elements G₁ or G₂. These may, for example, be guide pins (e.g., massive pins) that go into an opposite opening during the connection process.

Each of the four second plug-in connector parts V₁₁ and also V₂₁, V₂₂, V₂₃, V₂₄ is electrically connected in its turn to a corresponding local coil connection unit L₁, L₂, L₃ or L₄ n. In the connected state, the eight transmit signals provided by the power amplifier unit may be transmitted via the plug-in connector parts connected to one another to the respective local coil connection unit L₁, L₂, L₃ or L₄.

The local coil connection units L₁, L₂, L₃, L₄ are arranged on the patient table 17. If, for example, a local coil is connected and operated on the local coil connection unit L₄, the first plug-in connector part V₁₁ is plugged into the second plug-in connector part V₂₄ by the actuator unit A in order to electrically connect the transmit channels of the power amplifier unit to the local coil connection unit L₄.

Shown in FIG. 3 is a switching unit 26 with eight second plug-in connector parts V₂₁, V₂₂, V₂₃, V₂₄, V₂₅, V₂₆, V₂₇, V₂₈ that are arranged in two rows that are offset in the direction x₂ relative to one another. The eight second plug-in connector parts V₂₁, V₂₂, V₂₃, V₂₄, V₂₅, V₂₆, V₂₇, V₂₈ are each electrically connected to a local coil connection unit L₁, L₂, L₃, L₄, L₅, L₆, L₇, L₈. In one embodiment, the actuator unit is configured to also move the first plug-in connector part V₁₁ along the direction x₂ in order to be able to establish a connection with one of the second plug-in connector parts V₂₁, V₂₂, V₂₃, V₂₄, V₂₅, V₂₆, V₂₇ or V₂₈.

The local coil connection units L₁, L₂, L₃, L₄, L₅, L₆, L₇, L₈ are distributed, for example, at both ends of the patient table 17. In one embodiment, a local coil may be positioned at the right place for measurement more easily in this way even with a short cable length. Thus, a change of coils with re-plugging of the local coils is possible more easily.

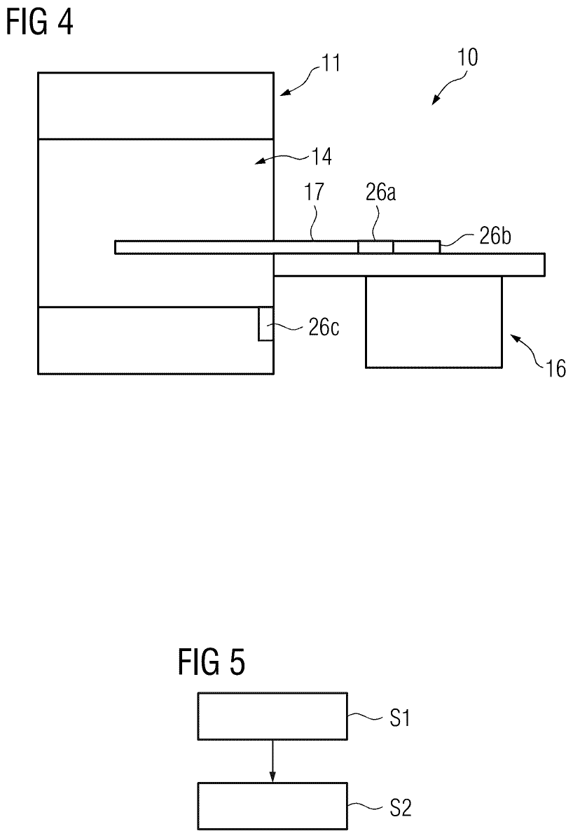

In FIG. 4, positions are shown that are particularly suitable for arrangement of the switching unit. These may thus be arranged, for example, at the end of the patient table 17, 26a, or behind the patient table, 26b, or on the front side of the magnet unit 11 behind its covering (e.g., when the transmit lines go from there into the table).

Shown in FIG. 5 is a method for providing a correct connection. In S1, the power amplifier unit provides a transmit signal to be emitted for creation of a radio-frequency pulse.

In S2, the amplitude and/or phase of a signal measured by a receive unit of the magnetic resonance apparatus (e.g., the local coil if the local coil is a transmit/receive coil) is evaluated. If the measured signal is too small, for example, it may be concluded from this that there was no correct connection made between power amplifier unit and transmit unit (e.g., of the local coil if the local coil is a transmit/receive coil). This may be caused, for example, by the switching unit not having made a correct connection.

S1 and S2 may take place at the beginning of carrying out a magnetic resonance sequence. The transmit signal to be emitted may have a small amplitude.

In conclusion, the magnetic resonance apparatus described in detail above as well as the method merely involve example embodiments that may be modified by the person skilled in the art in a wide diversity of ways, without departing from the field of the invention. Further, the use of the indefinite article “a” or “an” does not exclude the features concerned also being able to be present multiple times. Likewise, terms such as “unit” do not exclude the components concerned consisting of a number of subcomponents working together, which, where necessary, may also be spatially distributed.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A magnetic resonance apparatus comprising:
a power amplifier unit configured to provide transmit signals to be emitted;
a switching unit comprising an actuator unit;
a first plug-in connector part that is connected to the power amplifier unit; and
a number of second plug-in connector parts,
wherein the actuator unit is configured to move the first plug-in connector part relative to the number of second plug-in connector parts so that an electrical connection is established between the first plug-in connector part and one of the number of second plug-in connector parts.

2. The magnetic resonance apparatus of claim 1, wherein the actuator unit comprises at least one pneumatic drive, at least one hydraulic drive, or the at least one pneumatic drive and the at least one hydraulic drive.

3. The magnetic resonance apparatus of claim 1, wherein the actuator unit comprises at least one electric motor.

4. The magnetic resonance apparatus of claim 3, wherein the at least one electric motor is at least one screened electric motor.

5. The magnetic resonance apparatus of claim 3, further comprising a magnet unit operable for creation of a main magnetic field,
wherein the at least one electric motor comprises a rotor that acts together with the main magnetic field of the magnetic resonance apparatus as a permanent magnetic field of the at least one electric motor.

6. The magnetic resonance apparatus of claim 1, wherein the number of second plug-in connector parts are arranged in one plane, and
wherein the actuator unit is configured to move the first plug-in connector part parallel to the plane in order to bring the first plug-in connector part into a vicinity of one of the number of second plug-in connector parts.

7. The magnetic resonance apparatus of claim 6, wherein the actuator unit is configured to move the first plug-in connector part parallel to the plane in order to bring the first plug-in connector part into the vicinity of the one second plug-in connector part for alignment with the one second plug-in connector part.

8. The magnetic resonance apparatus of claim 1, wherein the number of second plug-in connector parts are arranged in one plane, and
wherein the actuator unit is configured to move the first plug-in connector part at right angles to the one plane in order to bring the first plug-in connector part into a releasable connection with one of the number of second plug-in connector parts.

9. The magnetic resonance apparatus of claim 1, further comprising:
a patient table; and
a magnet unit operable for creation of a main magnetic field of the magnetic resonance apparatus,
wherein the switching unit is arranged in an area of the patient table, in a vicinity of the magnet unit, or in the area of the patient table and in the vicinity of the magnet unit.

10. The magnetic resonance apparatus of claim 1, wherein the first plug-in connector part, the number of second plug-in connector parts, or the first plug-in connector part and the number of second plug-in connector parts comprise at least one coaxial electrical contact element, in particular at least one coaxial contact pin.

11. The magnetic resonance apparatus of claim 10, wherein the at least one coaxial electrical contact element is at least one coaxial contact pin.

12. The magnetic resonance apparatus of claim 1, wherein the first plug-in connector part, the number of second plug-in connector parts, or the first plug-in connector part and the number of second plug-in connector parts comprise at least one mechanical guide element, in particular at least one mechanical guide pin.

13. The magnetic resonance apparatus of claim 12, wherein the at least one mechanical guide element is at least one mechanical guide pin.

14. The magnetic resonance apparatus of claim 1, further comprising a system control unit,
wherein the actuator unit comprises an actuator control unit that is configured to receive control signals of the system control unit.

15. The magnetic resonance apparatus of claim 14, wherein the system control unit is configured to send the control signals to the actuator control unit depending on a magnetic resonance sequence to be carried.

16. The magnetic resonance apparatus of claim 1, further comprising a number of local coil connection units that are each configured to connect a local coil to the magnetic resonance apparatus,
wherein each of the number of second plug-in connector parts is electrically connected in each case to one of the number of local coil connection units.

17. The magnetic resonance apparatus of claim 1, further comprising at least one local coil that is configured to emit radio-frequency pulses in accordance with the transmit signals provided into an examination region of the magnetic resonance apparatus.

18. A method for ensuring a correct connection, the method comprising:
at the beginning of carrying out of a magnetic resonance sequence, providing, with a magnetic resonance apparatus, a transmit signal to be emitted for creation of at least one radio-frequency pulse, the magnetic resonance apparatus comprising a power amplifier unit configured to provide transmit signals to be emitted, a switching unit comprising an actuator unit, a first plug-in connector part that is connected to the power amplifier unit, and a number of second plug-in connector parts, wherein the actuator unit is configured to move the first plug-in connector part relative to the number of second plug-in connector parts so that an electrical connection is established between the first plug-in connector part and one of the number of second plug-in connector parts; and
evaluating a signal measured by a receive unit of the magnetic resonance apparatus.

19. The method of claim 18, wherein the measured signal is evaluated with respect to amplitude, phase, or amplitude and phase.
